(19) United States
(12) Patent Application Publication
(10) Pub. No.: US 2004/0161842 A1
(43) Pub. Date: Aug. 19, 2004

(54) Xenobiotic degradation in a partitioning bioreactor in which the partitioning phase is a polymer

(76) Inventors: Andrew J. Daugulis, Kingston (CA); Brian Amsden, Kingston (CA)

Correspondence Address: OGILVY RENAULT, 1981 MCGILL COLLEGE AVENUE, SUITE 1600, MONTREAL, QC, H3A2Y3, CA

(21) Appl. No.: 10/367,822

(22) Filed: Feb. 19, 2003

(51) Int. Cl. B09B 3/00; C12M 1/00

(52) U.S. Cl. 435/262.5; 435/289.1

(57) ABSTRACT

The present invention provides a process for the biodegradation of at least one xenobiotic. The process includes the steps of providing a bioreactor with an aqueous phase comprising water and at least one microorganism that is capable of metabolizing the xenobiotic and degradation products of the xenobiotic. The bioreactor is then provided with an organic phase comprising a solid or liquid polymer having an affinity for the xenobiotic and the xenobiotic is added to the bioreactor. The polymer is operable to absorb portions of the xenobiotic so that the aqueous concentration of the xenobiotic is substantially non-toxic to the microorganisms. The microorganisms degrade the xenobiotic in the aqueous phase causing the xenobiotic in the solid or liquid polymer to diffuse into the aqueous phase for degradation by the microorganisms.

### FIELD OF THE INVENTION

This invention relates to technology for the treatment of toxic organic pollutants, also known as xenobiotics. More particularly, the present invention relates to a process for the biodegradation of xenobiotics using a two-phase partitioning bioreactor in which the second (non-aqueous) phase is a solid or liquid polymer

### BACKGROUND OF THE INVENTION

The world's ecosphere continues to be challenged by the increasing amount and variability of toxic contaminants emitted by industrial activity. Such environmental contaminants are becoming more widespread as the pace of industrial activity accelerates, especially in developing countries, and also as a consequence of the difficulties in restricting the emissions of industrial activity from crossing national boundaries. The impact of these contaminants can be drastically acute (arising, for example, from industrial mishaps, such as in Bhopal India in 1984, and the accidental discharge of toxins), as well as longer term through chronic exposure. Indeed the link between immunological disorders (e.g. allergies and cancers) and environmental contamination is well-known. Environmental protection agencies in many countries have classified such compounds as priority, or high concern, pollutants, and their release is tightly regulated.

Among the most serious contaminants (both in terms of their impact and their resistance to treatment) are toxic organic compounds, particularly aromatic and halogenated compounds, and the subset of these known as xenobiotics. Xenobiotic compounds are materials that are invariably man-made, and are “foreign to nature” in the sense that they have been present in the ecosphere for relatively short time periods, such that efficient biodegradation pathways have not had adequate time to evolve. As a consequence, the biological treatment of these materials is particularly challenging due to the inhibition and/or toxicity of these compounds when they serve as microbial substrates.

In addition to their toxicity, many organic compounds are very poorly water soluble, which also decreases their capacity to be biodegraded. Polyaromatic hydrocarbons (PAHs) and polychlorinated biphenyls (PCBs) are examples of compounds that are highly toxic and/or carcinogenic, are priority pollutants, are very poorly soluble, and are, as a consequence, very difficult to degrade.

In the biological treatment of xenobiotic compounds, the most significant challenge is substrate delivery. That is, addition of the substrate at too high a concentration will inhibit or even kill the organisms, while substrate addition at too low a rate will cause the cells to starve, resulting in sub-optimal process performance. This situation is complicated by the fact that the substrate levels under consideration are extremely low; toxic levels of xenobiotic substrates can range from a few tens of milligrams per litre to a few hundred, making precise and controlled delivery of these materials exceedingly important. Conventional feedback control to achieve substrate delivery is not possible due to the lack of probes (to measure substrate levels, and compare to the desired setpoint value) for the specific substrates in question, and also because of the tendency of probes to drift, an unacceptable situation when miniscule variations in substrate concentrations can be lethal.

A recently-developed technology, the Two-Phase Partitioning Bioreactor or TPPB, has shown to be effective in the biodegradation of xenobiotic compounds (see for example the review in Daugulis, 2001, and Daugulis and Collins, 2001.). The TPPB concept is based on the use of a water-immiscible and biocompatible organic solvent that is allowed to “float” on the surface of a cell-containing aqueous phase. The solvent is used to dissolve large concentrations of xenobiotic substrates (this is usually readily achievable due to the very hydrophobic nature of most organic contaminants), a portion of which spontaneously transfer into the aqueous phase at low levels which are determined by the partition coefficient of the compound in question. Thus, although very high amounts of toxic organic substrates can be added to a bioreactor, the cells “see” only very low (sub-inhibitory) concentrations. Moreover, as cells consume some of the substrate, a disequilibrium is created which causes more of the xenobiotic substrate to be partitioned into the aqueous phase based on the system trying to maintain thermodynamic equilibrium. Thus, not only do appropriate amounts of xenobiotic substrates get delivered to the cells, but substrate delivery is also ongoing (until the organic phase becomes completely depleted) and the rate is determined by the metabolic activity of the cells. As the total cell concentration increases, or as the cells become more adapted to the inhibitory substrate, the increasing demand for substrate is met by equilibrium partitioning. TPPBs thus represent a system of cell-based process control in which the demand and supply of substrate are entirely “driven” by cellular processes.

The successful operation of a TPPB system for degrading xenobiotics requires careful selection of the organic solvent “delivery” phase. Among other criteria, the solvent phase should be: able to form a separate phase distinct from the aqueous phase, non-toxic to the degrading organisms in the bioreactor, non-bioavailable to the organism (i.e. not used as a substrate), non-volatile to prevent solvent losses should the system be aerated, inexpensive, and safe to operators. Although the two-liquid phase TPPB system has been shown to be effective at degrading xenobiotics, it has not always been possible to identify organic solvents that would meet all of these criteria in every instance.

### SUMMARY OF THE INVENTION

The present invention provides a novel process for the biodegradation of a xenobiotic or multiple xenobiotics in a two-phase partitioning bioreactor (TPPB) system in which the non-aqueous phase is a solid or liquid polymer.

The present invention provides a process for the biodegradation of a xenobiotic into degradation products comprising the steps of (i) providing in a bioreactor an aqueous phase comprising water and at least one microorganism capable of metabolizing at least one of the xenobiotics and degradation products thereof, (ii) providing in the bioreactor an organic phase comprising a solid or liquid polymer having an affinity for the xenobiotic relative to the aqueous solution and (iii) adding the xenobiotic to the bioreactor. The process optionally includes adding additional xenobiotic(s) when the xenobiotic(s) absorbed by the polymer has been partially diffused back into the aqueous solution and degraded by the microorganisms.

The present invention further provides a process for the biodegradation of at least one xenobiotic in a bioreactor having an aqueous phase, the process comprising the steps of processing a solid polymer such that it has an affinity for the target xenobiotics and to convert it into a desirable shape and size (e.g. small beads); (ii) adding the polymer to the aqueous phase of a bioreactor; (iii) adding at least one target xenobiotic to the bioreactor and allowing the polymer to absorb the xenobiotic to reduce the xenobiotic concentration to a level that will be substantially non-toxic to microorganisms; (iv) inoculating the bioreactor with one or more microorganisms capable of metabolizing the at least one xenobiotic or its degradation products thereby causing the xenobiotic in the aqueous phase to be degraded by the microorganisms and causing the xenobiotic contained in the polymer to diffuse into the aqueous phase to re-supply the degraded xenobiotic(s) until the concentration of xenobiotic is reduced to a predetermined level. In a further embodiment steps (iii) and (iv) are repeated.

In another of its aspects, the present invention provides a continuous process for the biodegradation of at least one xenobiotic comprising the steps of: (i) preparing and processing a polymer such that it has an affinity for the target xenobiotic(s) and is in a desirable shape and size; (ii) preparing an aqueous solution comprising water and one or more microorganisms capable of metabolizing the xenobiotic or its degradation products; (iii) adding the polymer to the aqueous phase of a bioreactor; (iv) adding at least one xenobiotic to the bioreactor, causing the polymer to absorb portions of the xenobiotic such that the aqueous concentration of the xenobiotic is substantially non-toxic to the organisms in the bioreactor; (v) causing the xenobiotic in the aqueous phase to be degraded by the microorganisms and causing the xenobiotic contained in the polymer to diffuse into the aqueous phase to re-supply the degraded xenobiotic; (vi) adding at least one further xenobiotic to the bioreactor, and repeating steps (v) and (vi).

In yet another of its aspects, the present invention provides a process for the biodegradation of at least one xenobiotic comprising the steps of: (i) contacting an aqueous phase containing a substantially toxic concentration of at least one xenobiotic with a liquid or solid polymer with an appropriate affinity, shape and size; (ii) causing the xenobiotic to diffuse into the polymer, thus reducing the aqueous xenobiotic concentration to substantially a non-toxic level; (iii) inoculating the aqueous phase with one or more microorganisms capable of metabolizing the xenobiotic or its degradation products, and/or utilizing indigenous organisms capable of metabolizing the xenobiotic; (iv) causing the microorganism(s) to degrade the xenobiotic contained in the aqueous phase, and that which diffuses from the polymer; (v) separating the polymer and reusing it according to steps (i) to (iv).

The present invention further provides a two-phase partitioning bioreactor for the biodegradation of at least one xenobiotic comprising a vessel containing an aqueous phase comprising water and at least one microorganism capable of metabolizing the xenobiotic and degradation products thereof and an organic phase comprising a solid polymer having an affinity for the xenobiotic.

### BRIEF DESCRIPTION OF THE DRAWINGS

The invention will be more clearly understood with reference to the attached description and the drawings in which:

FIG. 1 is a schematic diagram of an embodiment of the two-phase partitioning bioreactor (TPPB) of the present invention;

FIG. 2 is a graph that illustrates the mass fraction of benzene absorbed by EVA 40W;

FIG. 3 is a graph that illustrates the uptake of benzene from EVA beads and degradation by Alcaligenes xylosoxidans;

FIG. 4 is a graph that illustrates the mass fraction of phenol released from phenol-loaded EVA;

FIG. 5 is a graph that illustrates phenol uptake and delivery to Pseudomonas putida by EVA polymer spheres;

FIG. 6 is a graph that illustrates phenol uptake and delivery to Pseudomonas putida cells in batch fermentation experiments II and III;

FIG. 7 is a graph that illustrates the release and uptake of phenol by Pseudomonas putida in batch fermentation IV; and

FIG. 8 is a graph that illustrates the thermal history of, as-received (middle curve), used once (bottom curve) and used twice (top curve) EVA when subjected to heating from −400° C. to 100° C.

### DETAILED DESCRIPTION OF THE PREFERRED EMBODIMENTS

The delivery of a xenobiotic in the present invention preferably involves a two-phase partitioning bioreactor. Preferably, the two phases are an aqueous phase which contains, or into which is added, one or more microorganisms capable of degrading at least one target xenobiotic and its degradation products, and a solid polymer which has an affinity for the at least one xenobiotic, and which has been processed into an appropriate size and shape (e.g. small beads). It will now be understood that throughout the description the use of the term xenobiotic will refer to the use of one or more xenobiotic(s).

When the xenobiotic is present in, or is added to, the bioreactor, some of it will diffuse into the polymer, reducing the aqueous phase concentration. This concentration is intended to be non-toxic to the organisms. Moreover, in such a system, a xenobiotic equilibrium is established between the aqueous and polymer phases with the xenobiotic being partitioned from the polymer phase as the cells consume the xenobiotic in the aqueous phase. Thus, as cells consume the xenobiotic, more of it is transferred to the aqueous phase.

The polymer of the present invention is capable of absorbing portions of the xenobiotic such that the aqueous concentration of the xenobiotic is substantially non-toxic to the microorganisms and the microorganisms degrade the xenobiotic in the aqueous phase causing the xenobiotic in the solid or liquid polymer to diffuse into the aqueous phase for degradation by the microorganisms.

The solid polymer material should be properly selected or created. There are a number of useful criteria for polymer selection/creation. Preferably the polymer will readily form a separate phase from the aqueous phase. More preferably, it will not be water soluble and it will be non-bioavailable. More preferably, it will absorb and desorb the target xenobiotic of xenobiotics.

By proper selection/creation of the polymer and by its addition to the bioreactor in the proper amount, the aqueous phase xenobiotic concentration can be controlled to below levels that are toxic to the microorganisms. Examples of such polymers may be selected from those homopolymers, copolymers, terpolymers, block copolymers, triblock terpolymers, and star polymers, prepared from monomers which include but are not limited to ethylene, propylene, styrene, isobutylene, butadiene, isoprene, vinylacetate, vinylchloride, methyl methacrylate, ethylene terephthalate, dimethylsiloxane, diphenylsiloxane, diisocyanate, diols, diamines, dicarboxylic acids, caprolactam, acrylonitrile, 1-butene, and ethyl acrylate. Preferably, these polymers will be manipulated, as required, by means of cross-linking, blending, graft modification, and/or the formation of interpenetrating networks. The choice of monomer or monomers used and the manipulation of the structure of the polymer is not restricted and is within the realm of those skilled in the art.

In one preferred embodiment, the polymer will be made of EVA, and be made into beads that are less than 1 mm in size. See for example, the discussion and references cited in the Examples set out hereinbelow. It will be understood by a person skilled in the art that the polymer may be made into any desirable size and/or shape depending on the environment in which it is to be used. In another preferred embodiment, the polymer will be made of poly(styrene butadiene).

The choice of xenobiotic is not particularly restricted and is within the purview of a person skilled in the art. Preferably, the xenobiotic is an organic compound which may be unsubstituted, or substituted by a group such as halide, amino, cyano, and the like. In one embodiment, non-limiting examples of a suitable xenobiotic may be selected from the group consisting of benzene, toluene, ethylbenzene, xylene, styrene, polyaromatic hydrocarbons, phenol, pentachlorophenol, polychlorinated biphenyls, and mixtures thereof. In another embodiment, the xenobiotic is a nitroarene compound from the group consisting of RDX, HMX, TNT, and mixtures thereof. In yet another embodiment, the xenobiotic is a nerve and chemical warfare agent compound such as a member selected from the group tabun, sarin, GD, lewisite and adamsite, or mixtures thereof.

The mode of combining the polymer and the xenobiotic is not particularly restricted and is within the purview of a person skilled in the art. The polymer may be added to the aqueous phase of the bioreactor which already contains the xenobiotic, it may be added to the bioreactor prior to the addition of the xenobiotic, it may be used on a “spill” containing the xenobiotic and then added to the bioreactor, or it may be used to remove the xenobiotic from a gas stream prior to being added to the bioreactor. The concentration of the xenobiotic in the polymer is not particularly restricted provided that the resulting concentration of xenobiotic in the aqueous phase is substantially nontoxic to the microorganisms.

The choice of microorganisms is not particularly restricted provided that individual organism, or microbial consortia, can metabolize or biodegrade the xenobiotic(s) in question. Preferably, the microorganisms are selected from the genus comprising Pseudomonas, Arthrobacter, Sphingomonas, Mycobacterium, Alcaligenes, Klebsiella, Flavobacterium, Alteromonas, Enterobacter, Burkholderia, Escherichia, and mixtures thereof. Even more preferably, the microorganisms are selected from the group comprising Pseudomonas putida, Escherichia coli, Sphingomonas paucimobilis, and Sphingomonas aromativorans.

The mode of combining the microorganisms and water is not particularly restricted and is within the purview of a person skilled in the art. The microorganisms may arise from an inoculation of the bioreactor, or through a selective enrichment in which organism populations develop in response to the consumption of the xenobiotic. As the process is carried out the microorganisms will consume the xenobiotic which is partitioned from the polymer phase resulting in an increase in the population of microorganisms.

Of course those of skill in the art will recognize that the aqueous phase may further comprise one or more other additives (e.g. nutrients, solubilizing agents, surfactants and the like) to sustain the microorganisms and enhance their uptake of the xenobiotic.

Once prepared, the polymer, aqueous phase, microorganisms and xenobiotic are combined in a suitable reactor that may include provision for mixing, aeration, temperature control, pH control and the like.

When it is desired to operate the present process in batch or semi-batch mode (also known as “fed batch” or “sequential batch” mode), repeating the partitioning and biodegradation steps is repeated until the concentration of the xenobiotic is reduced to a predetermined value. Once the pre-determined value is reached, further xenobiotic is added to the bioreactor and the process may be restarted or continued.

Alternatively, the process may be conducted in a continuous mode wherein further xenobiotic is continuously added to the bioreactor as xenobiotic is partitioned to and biodegraded in the aqueous phase.

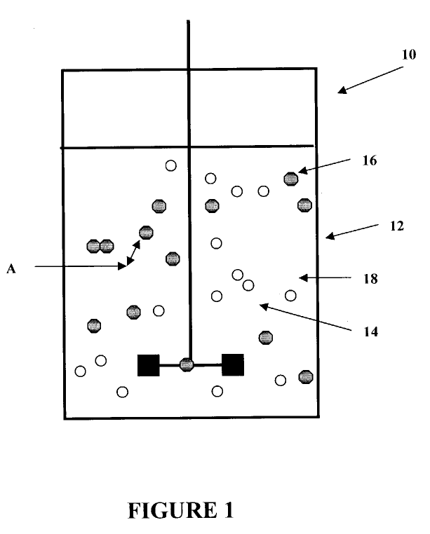

As discussed above, the present invention provides a two-phase partitioning bioreactor for the biodegradation of a xenobiotic. The two-phase bioreactor of the present invention will be more clearly understood with reference to FIG. 1 in which the bioreactor is indicated generally at numeral 10. The bioreactor 10 comprises a vessel 12 containing an aqueous phase 14 which comprises water and at least one microorganizm capable of metabolizing at least one of the xenobiotic and degradation products thereof and an organic phase comprising a solid polymer 16 having an affinity for the xenobiotic. The xenobiotic is absorbed by the polymer 16 and released to the cells 18 in the aqueous phase, indicated at arrow A.

Examples of the present invention will be illustrated with reference to the following Examples which should not be used to limit or construe the scope of the invention.

### EXAMPLE 1

ELVAX 40W—Poly(ethylene-co-vinyl acetate) (EVA 40W) macrospheres were used to absorb and desorb benzene from and to the aqueous phase in a bioreactor, reducing the benzene concentration to substantially non-toxic levels, and releasing it to cells for degradation. This polymer has the following characteristics: density, 0.965; melting point, 80° C.; effective radius, 0.17 cm; glass transition temperature −25° C.; structure, amorphous; vinyl acetate content, 40 mole %.

Absorption of the benzene by the polymer was able to reduce the aqueous concentration of benzene from substantially toxic to substantially non-toxic levels, allowing for the addition of microorganisms that degraded aqueous benzene. The concomitant reduction in benzene concentration in the aqueous phase thus caused (through maintaining thermodynamic equilibrium) benzene to be desorbed from the polymer, again allowing the organisms to degrade the substrate. In all, the cells ultimately degraded all of the benzene that had been added to the system, an amount that would otherwise have been toxic to the cells.

To determine the diffusivity of benzene in the polymer, benzene (105 mg/L) was injected using a 50 μL syringe into 13 250 mL-bottles (fitted with Teflon-coated silicone septa) each of which contained (aqueous): 7 g/L (NH₄)₂SO₄, 1 g/L MgSO₄, 8.42 g/L KH₂PO₄ and 80 μL/L trace element solution, stir bar, 3 g of EVA 40W and no headspace. The bottles were stirred vigorously and sampled for benzene content at various time intervals. Each bottle was sampled at one time only to ensure that there was no decrease in aqueous volume (and hence the introduction of a gas phase). It was assumed that the use of several bottles for sampling was comparable to continuous sampling from one large bottle without headspace.

For Batch Fermentation experiments, a Bioflo III Fermentor vessel (New Brunswick Scientific Co, Edison, N.J.) was filled with 3L of an aqueous solution comprised of: 7 g/L (NH₄)₂SO₄, 1 g/L MgSO₄, 8.42 g/L KH₂PO₄ and 80 μL/L trace element solution. This system was kept at 30° C., a pH of 6.6 (using 2 M KOH) and was agitated at 450 rpm. These conditions were maintained automatically by the Bioflo system during the experiment. Aeration consisted of headspace recirculation using a peristaltic pump, and 100-150 mL of pure O₂ was injected into the reactor every 30 minutes following inoculation. Using a 50 mL syringe, the amount of oxygen injected depended on the amount that was needed in order to keep the % of oxygen saturation at a high and steady level.

Following startup, 4.1 mL (3628 mg) of benzene were added to the aqueous medium in the reactor. Aqueous samples were drawn periodically to determine the concentration of benzene. When the concentration approached a constant value due to equilibration of benzene with the reactor headspace, 310 g of EVA (sterilized using UV) were added to the reactor to reduce the benzene concentration to substantially non-toxic levels. When the concentration ceased to change, the reactor was inoculated with Alcaligenes xylosoxidans. The A. xylosoxidans cells had been precultured at 30° C. on a shaker in 14 125 mL-flasks containing 50 mL aqueous volume of: 2 g/L sodium benzoate, 7 g/L (NH₄)₂SO₄, 1 g/L MgSO₄, 8.42 g/L KH₂PO₄, 6.6 g K₂HPO₄, and 80 μL/L of trace element solution. After 25 hours of incubation, the contents of the inoculation flasks were centrifuged (to reduce the total volume of the inoculum to 140 mL) and added to the bioreactor

Bacterial concentration and benzene concentration were monitored periodically throughout this experiment. Benzene concentration was measured by gas chromatography (GC). Aqueous samples (2.5 mL) were injected into 5 mL vials (fitted with Teflon coated silicon septa) containing 2.5 mL of n-hexadecane 99%. A partition coefficient of 142 was assumed (Yeom and Daugulis, 2000). The samples were incubated at 30° C. before being injected (injection volume of 0.2 μL) into the GC. Benzene concentration was determined from the peak area by comparison to a previously determined calibration curve. Samples containing bacteria were centrifuged for 10 minutes (at 3340 rpm, 30° C.) before GC analysis. Bacterial cell concentration was determined by measuring the optical density of the sample using an Ultrospec 3000 Spectrophotometer at 640 nm and comparing it to a calibration curve.

The diffusivity of benzene in EVA 40W was determined by converting benzene concentrations (obtained at various times during the diffusivity experiment) into mass fractions and plotting them against time. The resulting curve was then modeled using the following equation:

$$\frac{{M}_{t}}{{M}_{∞}} = 1 - \frac{6}{{Π}^{2}} {\sum }_{n = 1}^{∞} {( - 1 )}^{n} exp ( \frac{- {n}^{2} {D}_{e} t Π}{{r}^{2}} ) \qquad ( 1 )$$

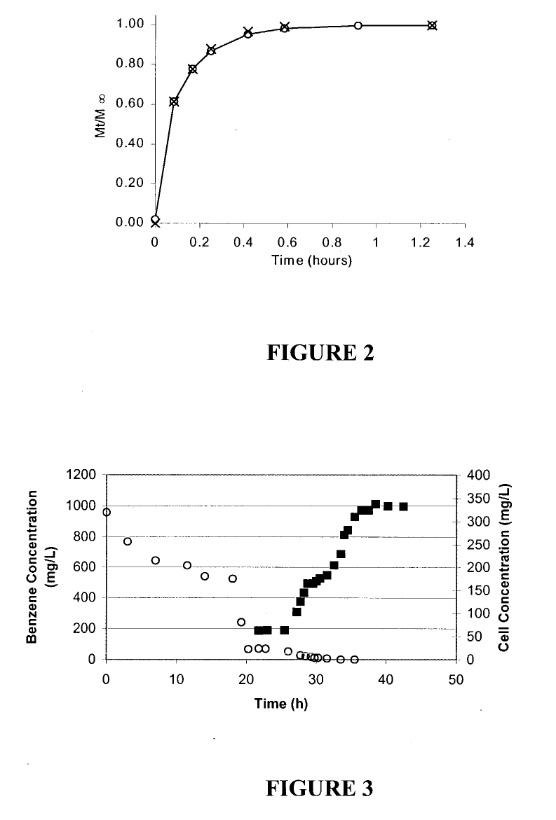

The solute diffusivity coefficient, Dₑ, was iterated until the closest possible fit between the two curves was obtained (lowest Sum of Squared Residuals—SSR). This fit is shown in FIG. 2 in which “o” represents the fit and “x” the experimental. A diffusivity of 4.7×10⁻⁶ cm²/s resulted in the lowest SSR of 0.01 and 1 of EVA 40W absorbed 4.19 mg of benzene.

A demonstration of concept of the process of benzene uptake by polymer and release for degradation is depicted in FIG. 3. A total of 4.1 mL (3628 mg) of benzene was injected into the vessel and the contents were allowed to reach equilibrium with the headspace of the bioreactor. After 18 hours, the aqueous benzene concentration was ˜525 mg/L (a concentration that is substantially toxic to the cells), at which time 310 g of EVA 40W were added. In two hours, the polymer beads had reduced the aqueous concentration of benzene to ˜52 mg/L, which is substantially non-toxic to the microorganisms. The system was then inoculated with A. xylosoxidans. Approximately four hours after inoculation, the cell (▪) and benzene (o) concentrations began to change as shown in FIG. 3. Benzene concentration decreased until there was no detectable benzene in the fermentation broth in less than 12 hours (from the time of inoculation). Cell concentration increased for approximately 15 hours after inoculation leveling off at a concentration of 324 mg/L.

EVA 40W macrospheres were thus successfully used to absorb benzene from the aqueous phase, rendering it substantially non-toxic, and to deliver it to A. xylosoxidans in a bioreactor. Twelve hours after inoculation all of the initial benzene added had been degraded.

### EXAMPLE 2

The bacterium used in this study was Pseudomonas putida (ATCC 11172), which degrades phenol via the aerobic meta-cleavage pathway (Collins and Daugulis, 1996). P. putida cells are rod-shaped, gram negative bacteria that require a phenol concentration of up to approximately 400-500 mg/L for optimum growth (Vrionis et al., 2001). Concentrations above 800 mg/L are substantially toxic to these organisms.

ELVAX—poly(ethylene vinyl acetate) (EVA 40W) macrospheres were used for phenol delivery. Synthetic media consisting of mineral salts, trace elements and iron chloride were used in all of the experiments. Phenol and dextrose were used as carbon sources for various experiments.

All phenol samples were analyzed by the 4-aminoantipyrine method (Vrionis et al., 2002). Absorbance readings were obtained using an Ultrospec 3000 spectrophotometer at 505 nm. Bacterial cell concentration was determined by measuring the optical density of the sample using the Ultrospec 3000 Spectrophotometer at 640 nm and comparing it to a calibration curve.

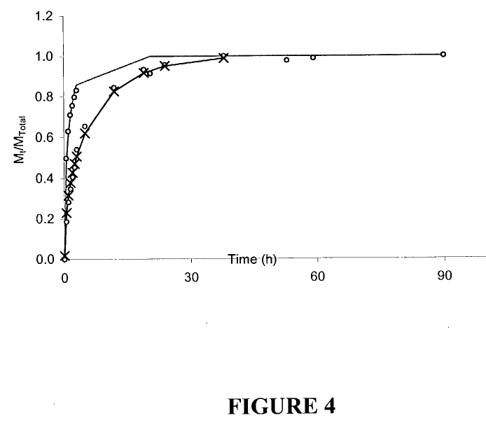

Two Preliminary Desorption Experiments were conducted. EVA 40W macrospheres were added to a concentrated phenol solution and allowed to stand for 3 hours. After 3 hours, the EVA 40W was filtered, washed briefly with methanol and allowed to air dry for 24 hours. In the first experiment (1^{st} run), a flask containing 2.004 g of phenol-loaded EVA 40W and 40 mL of medium (for Desorption Experiments) was placed on a shaker at 30° C. This medium was replaced periodically with fresh medium and analyzed for phenol content. The second experiment (2^{nd} run) consisted of reloading the EVA 40W from the first desorption experiment and repeating the process (1.257 g of phenol-reloaded EVA 40W was used). Phenol concentrations at various time intervals were converted to mass fractions and plotted against time. The curve given by equation (1) was fitted to the experimental curve and the diffusivity coefficient, Dₑ, was iterated until the closest possible fit was obtained (lowest Sum of Squared Residuals—SSR). A diffusivity coefficient of 2.2×10⁻³ cm²/h produced the best fit (SSR=0.01) between the experimental data from the 1^{st} run, represented by “o” in FIG. 4, EVA 40W and the curve generated by the desorption equation. This relationship is presented in FIG. 4 along with the curve for the reused (2^{nd} run), represented by “x”, EVA 40W. After 20 hours, 91% of the phenol had desorbed out of the 1^{st}-run EVA 40W. The total amount of phenol released was 1.02 g from 2.04 g of EVA 40W (0.51 g phenol/g EVA 40W). The 2^{nd}-run EVA data yielded a diffusivity coefficient of 4.8×10⁻⁴ cm²/h (SSR=0.01). In 20 hours, 94% of the phenol was released. The total mass of phenol released was 0.42 g/g EVA 40W. For the 1^{st} and 2^{nd} (run) EVA 40W, an effective radius of 0.21 cm was estimated based on the increased mass of the EVA 40W from phenol loading.

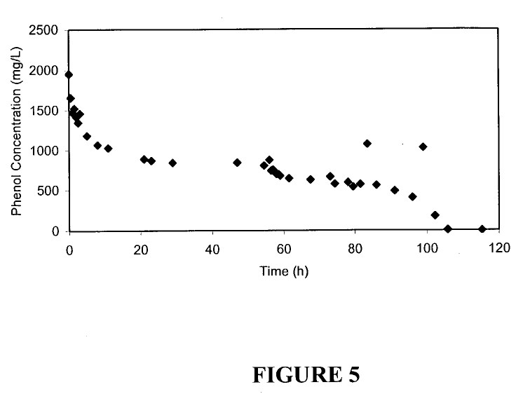

For phenol release/degradation studies, a Bioflo III Fermentor vessel (New Brunswick Scientific Co, Edison, N.J.) was filled with 2800 mL of bioreactor medium at a phenol concentration of 2000 mg/L, and sterilized. For Batch Fermentation I an arbitrarily chosen amount of EVA 40W, 198.3 g, was sterilized with UV radiation and added to the reactor. The reactor was agitated at 400 RPM, and kept at 30° C. and a pH of 6.9 (using 2M NaOH). These conditions were maintained automatically by the Bioflo system throughout the duration of the experiment. A condenser was used to prevent volatilization of the reactor contents. Samples were taken for 24 hours (at different time intervals) to monitor the uptake of phenol by the EVA 40W macrospheres. FIG. 5 shows that the concentration of phenol decreased to a steady concentration of 845 mg/L in ˜20 hours. This corresponded to a reduction of 0.016 g phenol/g of EVA 40W and indicated that 93 g of EVA 40W had to be added to the system to further reduce the concentration to sub-inhibitory levels of approximately 542 mg/L. An additional 93 g of EVA 40W was therefore added and the concentration of phenol was reduced to 578 mg/L 27 hours after addition. Since the phenol concentration was below 800 mg/L, the bioreactor was inoculated at 73 hours. The system was aerated at 3 vvm (air volume/medium volume per minute). Samples of the bioreactor broth were collected for phenol analysis at various times during the fermentation.

The phenol concentration remained relatively constant for approximately 10 hours after inoculation and then it decreased to 0 g/L in approximately 20 hours, as shown in FIG. 5. There was considerable foaming and cell growth on the walls of the bioreactor making it impossible to accurately monitor the cell concentration. The reactor contents began to turn yellow approximately 16 hours after inoculation, indicating the presence of a degradation intermediate.

To determine whether the polymer beads still contained any phenol after the release/degradation study, the EVA 40W used in the first fermentation was placed in a flask containing 100 mL of medium. These contents were agitated on a shaker for 24 hours at 30° C. The medium was then analyzed for phenol content. The concentration was found to be 0 g/L, indicating that all of the phenol had been released from the polymer beads.

Three additional release/degradation studies, Batch Fermentations II, III and IV were conducted in a manner similar to that in Batch Fermentation I. This time, however, the total amount of EVA 40W required to reduce the aqueous phenol from toxic levels (2000 mg/L) to non-toxic levels (800 mg/L), 291.3 g (430 mL), was added to the reactor at the beginning of the experiment. Phenol concentrations were monitored just as in the previous experiment. The fermentor was inoculated after 24 hours. Samples of the medium were taken throughout the fermentation to monitor phenol, as well as cell concentration. Antifoam (˜0.5 mL in total) was added manually to the reactor contents when foam was starting to form. Aeration was maintained at 1 vvm to reduce foaming. Dissolved oxygen was monitored via a dissolved oxygen probe.

Batch Fermentation IV consisted of reusing EVA from Batch Fermentation III. Aside from the used EVA 40W, this experiment was exactly the same as Batch Fermentation III.

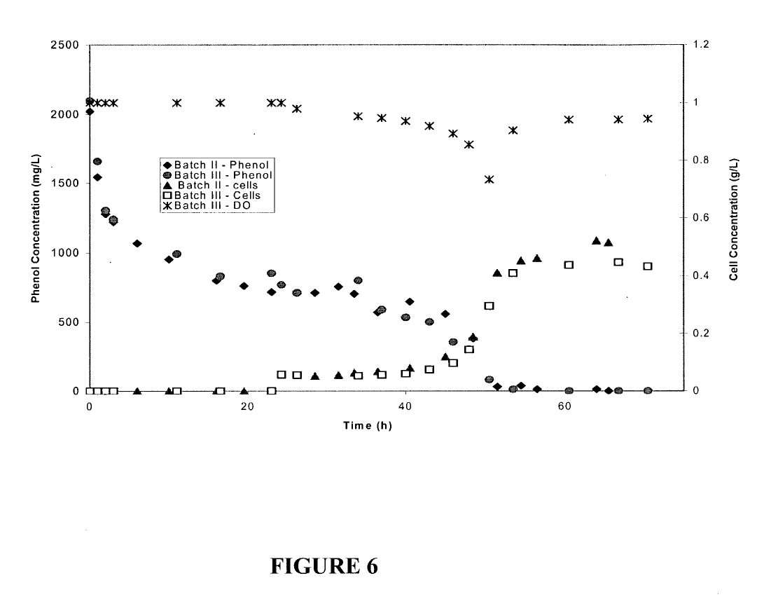

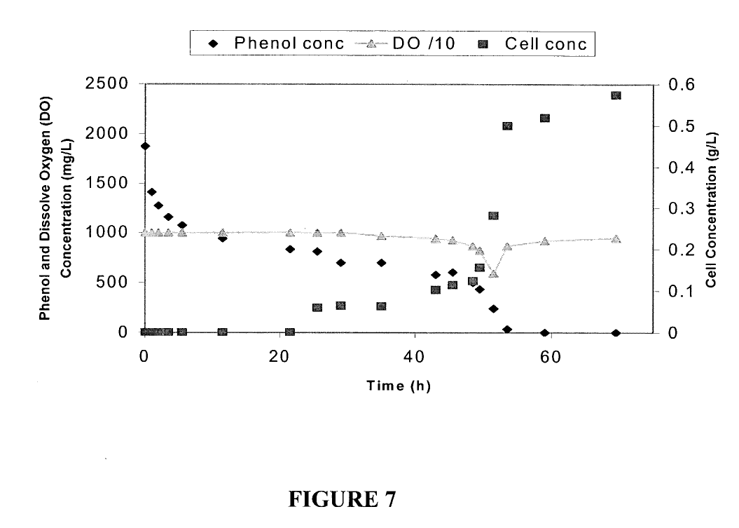

For Batch Fermentations II, III and IV, the following conditions applied: initial phenol concentration, 2000 mg/L; EVA 40W added, 291.3 g, seeded with 200 mL of P. putida-containing inoculum 24 hours after addition of EVA 40W macrospheres. Phenol concentrations for batch experiments II & III (shown in FIG. 6) and IV (shown in FIG. 7) were monitored through out the fermentations. In Batch Fermentation II the phenol concentration decreased to 718 mg/L (at t=20 hours) after addition of EVA 40W. After inoculation, the phenol concentration remained constant for approximately 13 hours (until t=37 hours) after which it started to decline exponentially, reaching a final concentration of 0 g/L (t=˜60 hours). FIG. 6 illustrates the phenol concentration in the bioreactor at various times in the experiment.

Cell concentration was measured periodically after inoculation and is also shown in FIG. 6. During the first 13 hours (after inoculation), this concentration remained relatively unchanged. Approximately 45 hours after addition of EVA 40W, the cell concentration increased dramatically from 0.12 g/L to 0.41 g/L in a matter of 6.5 hours. This concentration increased only slightly afterwards until it reached a final concentration of 0.51 g cells/L. Some cells were growing on the bioreactor wall.

Batch Fermentation III had a phenol concentration of 712 mg/L (at t=˜20 hours and) at the time of inoculation. Cell and phenol concentrations remained constant for approximately 13 hours after inoculation and changed drastically over a period of about 10 hours as shown in FIG. 6. There was some foaming present at t=50 hours and the final cell concentration was 0.43 g/L. All 5.6 g of phenol were degraded in ˜30 hours (after inoculation). Dissolved oxygen level (%) in the bioreactor was monitored as well and showed a significant drop during the 10-hour period of greatest phenol and cell concentration change (FIG. 6).

Batch Fermentation IV was conducted in the same manner as Batch Fermentations II and III. The polymer from Batch Fermentation III was reused in Batch Fermentation IV. P. putida was introduced to the system at a phenol concentration of 756 mg/L. After inoculation, the phenol concentration remained relatively constant for about 16 hours after which it decreased as illustrated in FIG. 7, until it reached a final phenol concentration of 0 g/L (approximately 36 hours after inoculation). The cell concentration started to increase after about 16 hours (after inoculation) and showed a rapid increase from approximately 24 to 29.5 hours (t=48 to 53.5 hours) after inoculation and approached a final concentration of 0.57 g cells/L. Dissolved oxygen data showed a minimum 27.5 hours after inoculation (FIG. 7). In all of the fermentations, the medium appeared yellow when the cell concentration started to increase (t=˜43 hours for fermentations II-IV), indicating the presence of a phenol degradation intermediate.

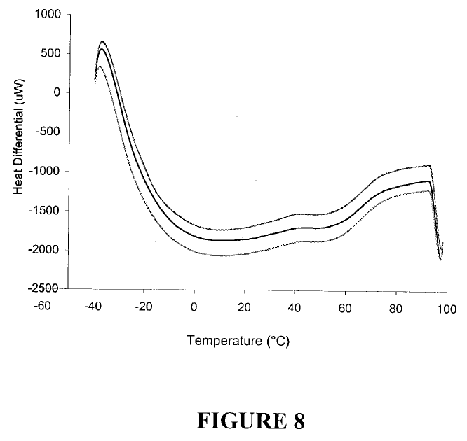

EVA 40W used once (Batch Fermentation III) and twice (Batch Fermentation IV) to absorb and then release phenol was analyzed for changes in thermal response when it was heated from −40° C. to 100° C. at a uniform rate of 10° C./min using a Seiko SSC/5200 Differential Scanning Calorimeter (DSC). These thermal histories were compared to those of fresh EVA 40W and are illustrated in FIG. 8. There were no differences in either samples of EVA 40W in comparison to the fresh EVA 40W. This result indicated that there was no detectable phenol remaining entrapped within the EVA, i.e. all the originally absorbed phenol had been desorbed during the degradation process.

To summarize the results from Example 2, the phenol desorption experiments were conducted to determine if EVA can be used to absorb phenol from an aqueous solution reducing the concentration to substantially non-toxic levels, and to deliver it to bacteria for degradation. In Batch Fermentations I, II and III, the phenol concentrations were decreased to 578 mg/L, 718 mg/L and 712 mg/L respectively, from 2000 mg/L, by the addition of the EVA polymer beads. This corresponds to a 71% reduction in phenol concentration for Batch fermentation I, and a 62% reduction in phenol concentration for Fermentations II and III. A 60% phenol reduction was achieved when the EVA was reused in the fourth bioreactor run and the concentration was reduced to approximately 756 mg/L. Based on data from runs II, III and IV, 1 g of EVA removed ˜0.014 g of phenol. The results demonstrate that EVA 40W is an effective “sponge” for phenol even when reused. In all, 5.6 g of phenol were degraded in approximately 30 hours (after inoculation) in each of the Batch Fermentations.

Some of the macrospheres from Batch Fermentation III were placed in fresh medium which was checked for phenol content after 24 hours on a shaker. No phenol was detected in the medium, which suggests that the macrospheres were emptied in the bioreactor (III). In addition, DSC analyses showed that the polymer used in the fermentations (once and two times) had the same thermal history as the fresh polymer further verifying that no measurable phenol remained in the EVA.

The non-biodegradable polymer (EVA 40W) used in this study was successful in delivering phenol at a controlled rate to microorganisms in a bioreactor. The release of phenol was controlled by the metabolic activity of P. putida and resulted in the complete degradation of phenol. When the polymer was re-used, the same results were obtained as with the as-received EVA 40W suggesting that this system can be used repeatedly. The non-degradable polymer technology in this study presents an effective alternative to the organic liquid solvent used in earlier TPPBs. The solid solvent is not subject to volatilization and is not bioavailable to the microbes. It allows for rigorous mixing in the bioreactor and can be applied in fluidized bed systems.

Those skilled in the art will recognize variants of the embodiments described herein and presented in the above Examples. Such variants are intended to be within the scope of the invention and are covered by the appended claims.

### REFERENCES

Collins, L. D. and Daugulis, A. J., Use of a Two Phase Partitioning Bioreactor for the Biodegradation of Phenol, Biotechnology Techniques, 10, 643-648, (1996).

Daugulis, A. J., Two-Phase Partitioning Bioreactors: A New Technology Platform For Destroying Xenobiotics, Trends in Biotechnology, 19, 459-464 (2001).

Daugulis, A. J. and Collins, D. L., Process for Biodegradation of a Xenobiotic, U.S. Pat. No. 6,284,523 (2001).

Vrionis, H., Kropinski, A. M. B. and Daugulis, A. J., Enhancement Of A Two-Phase Partitioning Bioreactor System By Catalyst Modification: Demonstration Of Concept, Biotechnol. Bioeng. 79, 587-594 (2002).

Yeom, S. H. and Daugulis, A. J., Development of a Novel Bioreactor System for the Treatment of Gaseous Benzene, Biotechnol. Bioeng, 72, 156-165 (2001).

1. A process for the biodegradation of at least one xenobiotic into degradation products thereof comprising the steps of:
(i) providing in a bioreactor an aqueous phase comprising water and at least one microorganism capable of metabolizing the at least one xenobiotic and degradation products thereof;
(ii) providing in the bioreactor an organic phase comprising a solid or liquid polymer having an affinity for the at least one xenobiotic and capable of absorbing and desorbing at least a portion of the xenobiotic; and
(iii) adding the at least one xenobiotic to the bioreactor for degradation thereof.

2. The process according to claim 1 further comprising repeating step (iii) when the xenobiotic in the solid or liquid polymer has been partially diffused.

3. The process according to claim 1 wherein the polymer is water insoluble.

4. The process according to claim 1 wherein the polymer is non-bioavailable.

5. The process according to claim 1 wherein the microorganism is selected from the group consisting of Pseudomonas, Arthrobacter, Sphingomonas, Mycobacterium, Alcaligenes, Klebsiella, Flavobacterium, Alteromonas, Enterobacter, Burkholderia and Escherichia.

6. A process for the biodegradation of at least one xenobiotic into degradation products thereof comprising the steps of:
(i) contacting an aqueous phase containing at least one xenobiotic with a solid or liquid polymer, the polymer being capable of absorbing and desorbing at least a portion of the at least one xenobiotic, and allowing the diffusion of at least a portion of the xenobiotic therein;
(ii) inoculating the aqueous phase with at least one of an indigenous organism capable of metabolizing the at least one xenobiotic, and at least one microorganism capable of metabolizing the at least one xenobiotic and degradation products thereof; and
(iii) separating the polymer from the aqueous phase for reuse.

7. The process according to claim 6 further comprising forming the polymer to a predetermined size and shape prior to step (i).

8. The process according to claim 6 wherein the polymer is water insoluble.

9. The process according to claim 6 wherein the polymer is non-bioavailable.

10. The process according to claim 6 wherein the microorganism is selected from the group consisting of Pseudomonas, Arthrobacter, Sphingomonas, Mycobacterium, Alcaligenes, Klebsiella, Flavobacterium, Alteromonas, Enterobacter, Burkholderia and Escherichia.

11. A two-phase partitioning bioreactor, for the biodegradation of a xenobiotic, comprising:
a vessel containing an aqueous phase comprising water and at least one microorganizm capable of metabolizing the at least one xenobiotic and degradation products thereof; and
an organic phase comprising a solid or liquid polymer having an affinity for the xenobiotic and capable of absorbing and desorbing at least a portion of the at least one xenobiotic.
